(19) United States
(12) Patent Application Publication
(10) Pub. No.: US 2017/0014069 A1
(43) Pub. Date: Jan. 19, 2017

(54) SPECTRAL IMAGING BASED FLUID VOLUME MAP

(71) Applicant: KONINKLIJKE PHILIPS N. V., Eindhoven (NL)

(72) Inventors: Raz CARMI, Haifa (IL); Galit Sarit KAFRI, Newton, MA (US); Mark Eric OLSZEWSKI, Solon, OH (US)

(21) Appl. No.: 14/798,682

(22) Filed: Jul. 14, 2015

(51) Int. Cl. A61B 5/00; A61B 5/0295; A61M 5/00

(52) U.S. Cl. CPC A61B 5/489; A61M 5/007; A61B 5/418; A61B 5/0295; A61B 5/742; A61B 2576/00; A61B 2090/374

(57) ABSTRACT

A method includes obtaining image data that includes contrast enhanced spectral image data from a contrast enhanced spectral volume scan of a subject. The method further includes obtaining images that include a set of localized dynamic contrast enhanced images from a localized dynamic contrast enhanced series scan of the subject. The method further includes determining a fluid volume map based on the image data and the images. A computing system includes a computer readable storage medium with computer readable instructions for a fluid volume map determiner. The instructions determine a volumetric fluid volume map based on contrast enhanced spectral volume image data from a contrast enhanced spectral volume scan and a set of localized dynamic contrast enhanced images from a localized dynamic contrast enhanced series scan. The computing system further includes a processor that implements the instructions and determines the volumetric fluid volume map.

The following generally relates to fluid (e.g., blood, lymph, etc.) volume maps, and more particularly to creating one or more fluid volume maps based on contrast enhanced spectral volume image data from a contrast enhanced spectral volume scan and a set of localized dynamic contrast enhanced (localized-DCE) images from a localized dynamic contrast enhanced series scan, and is described with particular application to computed tomography (CT). However, the following is also amenable to other imaging modalities such as magnetic resonance (MR), positron emission tomography (PET), and/or other imaging modalities.

In medical diagnostic imaging, obtaining information on the blood circulation within the vessels and tissues of interest for several common diseases can facilitate diagnosing a disease. For example, such information is relevant to brain imaging in the context of stroke, cardiac imaging, cancer staging and monitoring, and pulmonary diseases. Typically, several particular blood flow characteristics such as perfusion (flow per volume), blood volume, permeability, time-to-peak, and mean transient time are determined.

There are known relations between these parameters. For example, perfusion and blood volume are usually strongly correlated. The blood flow characteristics are calculated from image data of contrast enhanced scans such as iodine in CT, Gadolinium in MRI, or radiotracer in nuclear medicine. However, there are known limitations to these techniques such as high radiation dose due to continuous or repeated CT scans, limited axial coverage, and practical inaccuracies due to patient motion and breathing.

Spectral CT such as dual energy or photon counting scanners can quantitatively detect iodine contrast agent (or other material(s)) with high sensitivity. Generally, since the absorption of a photon by a material is dependent on the energy of the photon traversing the material, the detected radiation also includes spectral information, which provides additional information indicative of the material composition of the scanned material. A spectral CT scan captures these spectral characteristics.

Due to the iodine high photoelectric effect and k-edge attenuation at 33.2 keV, iodine can be differentiated from other materials and tissues in the human body. Therefore, an iodine map can be generated for the scanned volume. Such a map can quantify the temporary amount of contrast agent in a tissue in a particular instance of scan, but the quantitative blood volume maps still cannot be derived just from such single scan since the contrast agent enhancement changes rapidly with the blood circulation.

Moreover, the correct normalization relative to the time attenuation curve (TAC) of a main artery is usually required to compensate on varying effects such as contrast agent concentration, injection pattern and cardiac output. The contrast agent in the reference main artery changes in time as well. Combining spectral CT scanning with dynamic contrast enhanced perfusion CT scanning, unfortunately, still requires high radiation dose due to the multiple diagnostic scan frames.

Aspects described herein address the above-referenced problems and others.

In general, the aspects include generating fluid volume maps based on contrast enhanced spectral volume image data and a set of localized-DCE images. The localized-DCE images generally provide limited information with respect to volumetric diagnostic DCE. In one non-limiting instance, with respect to volumetric quantitative blood volume maps, this entails calculating an approximated blood flow model for an organ(s) of interest based on the localized-DCE images and contrast injection parameters of the volume scan, calculating iodine maps from the volume image data, and generating the volumetric quantitative blood volume maps based on the approximated blood flow model and the iodine maps.

In one aspect, a method includes obtaining image data that includes contrast enhanced spectral image data from a contrast enhanced spectral volume scan of a subject. The method further includes obtaining images that include a set of localized-DCE images from a localized-DCE series scan of the subject. The method further includes determining a fluid volume map based on the image data and the images.

In another aspect, a computing system includes a computer readable storage medium with computer readable instructions for a fluid volume map determiner. The instructions determine a volumetric fluid volume map based on contrast enhanced spectral volume image data from a contrast enhanced spectral volume scan and a set of localized-DCE images from a localized-DCE series scan. The computing system further includes a processor that implements the instructions and determines the volumetric fluid volume map.

In another aspect, a computer readable storage medium is encoded with computer with readable instructions, which, when executed by a processor of a computing system, causes the processor to: determine a volumetric blood volume map based on contrast enhanced spectral volume image data generated in response to contrast enhanced spectral volume scan of a subject and images and a set of localized-DCE images generated in response to a localized-DCE series scan of the subject

The invention may take form in various components and arrangements of components, and in various steps and arrangements of steps. The drawings are only for purposes of illustrating the preferred embodiments and are not to be construed as limiting the invention.

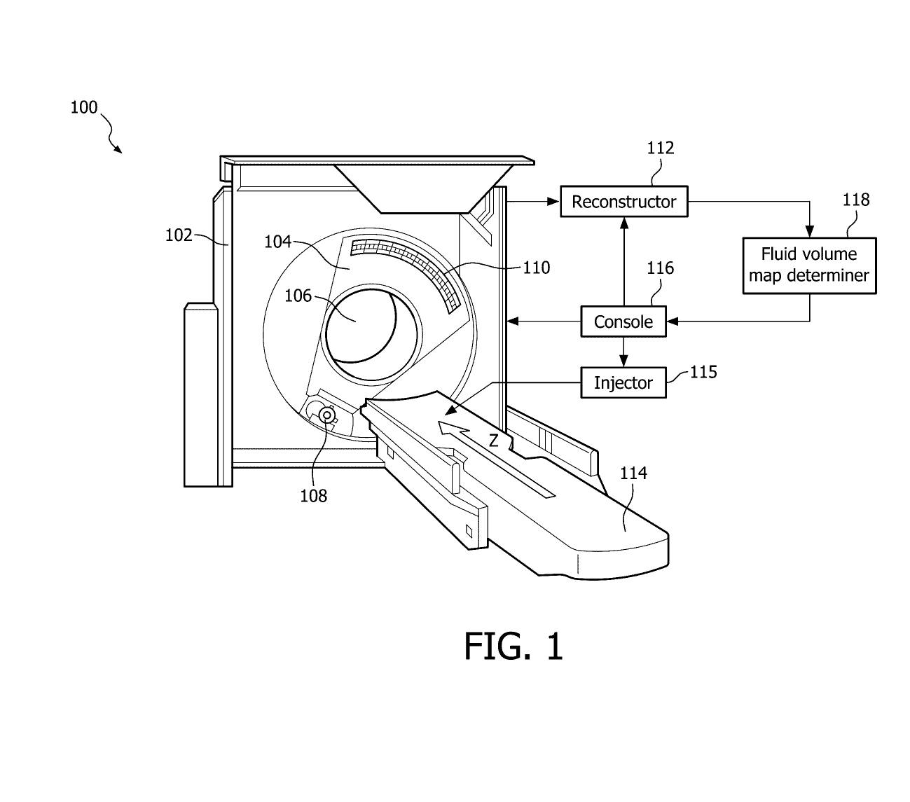

FIG. 1 schematically illustrates a fluid volume map determiner in connection with an example imaging system configured for conventional and spectral imaging.

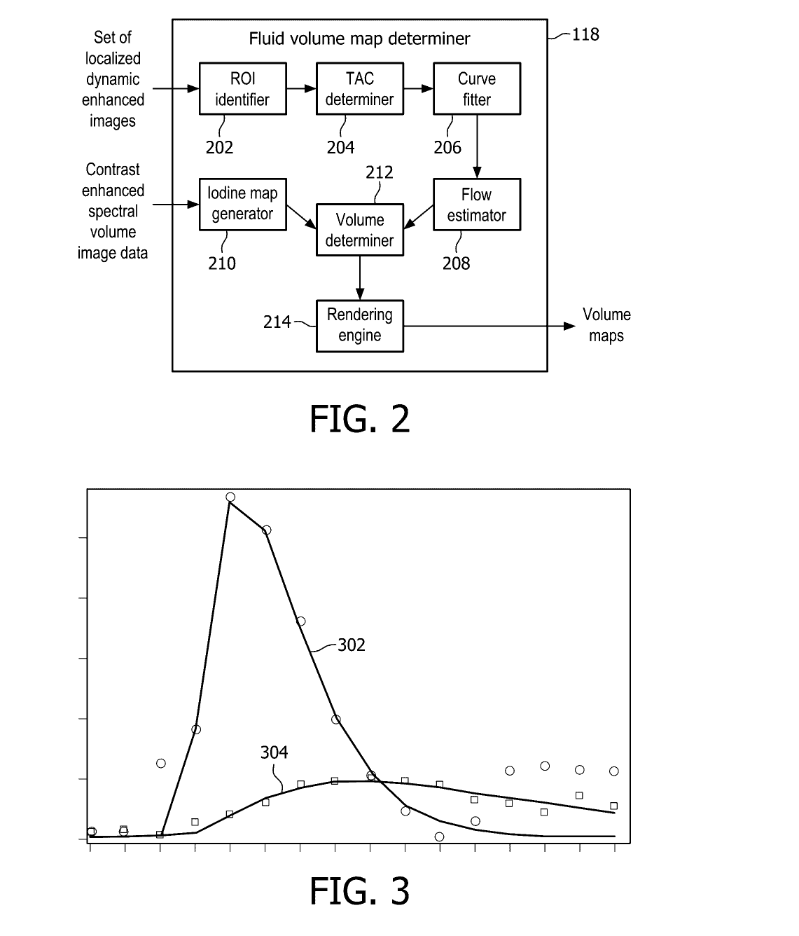

FIG. 2 schematically illustrates an example of the fluid volume map determiner.

FIG. 3 shows an example fit of gamma-variate curves to measured TAC curves of ROIs for the aorta and the spleen.

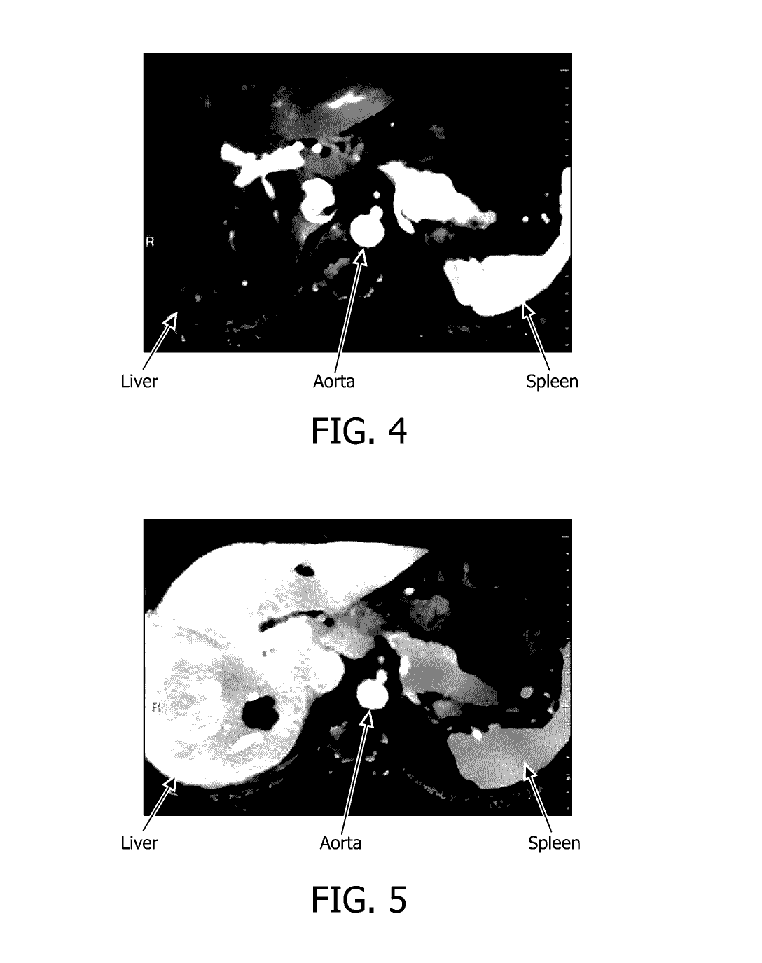

FIG. 4 shows spectral image data based iodine maps of a dual-circulation organ (liver) and a single-circulation organ (spleen) at a first time.

FIG. 5 shows spectral image data based iodine maps of a dual-circulation organ (liver) and a single-circulation organ (spleen) at a second time.

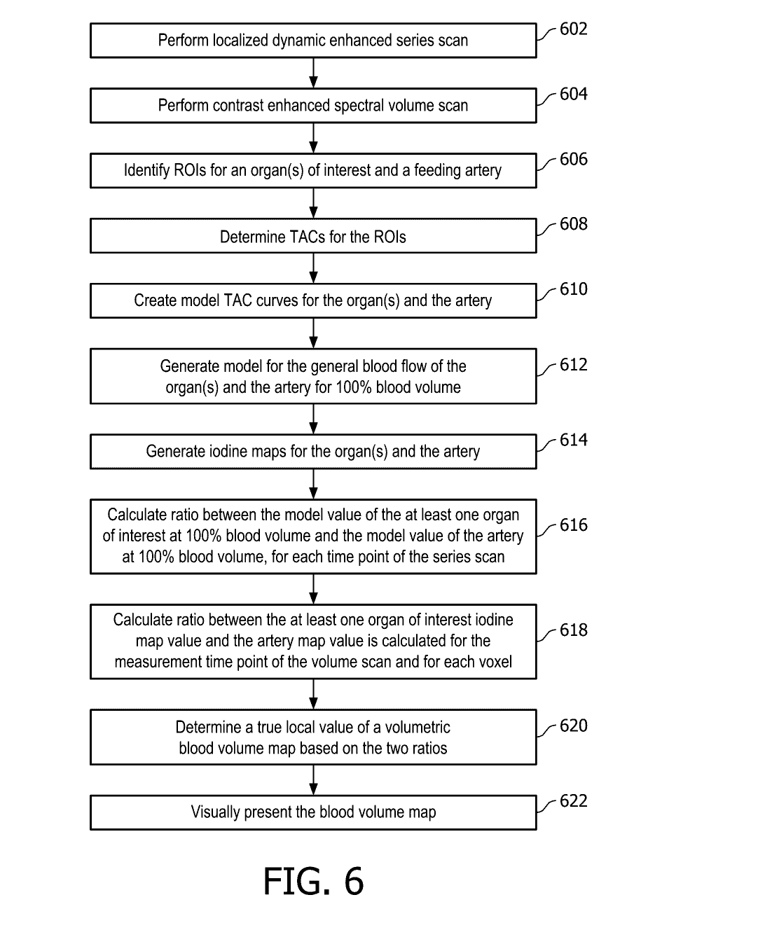

FIG. 6 illustrates a method for determining a fluid volume map based on contrast enhanced spectral volume image data and a set of localized-DCE images.

The term ‘blood volume’ or ‘fluid volume’ herein refers to the relative volume of the relevant fluid (e.g. in percentages) within an overall unit volume such as image voxel or another determined volume of interest. The term ‘localized-DCE’ refers to a scan that provides information on the fluid dynamics and distribution in only a subset of local (or confined) regions such as ROIs on a vessel and/or on an organ tissue. In contrast, the term ‘volumetric-DCE’ refers to a scan that provides information on the fluid dynamics and distribution in each voxel within a clinically relevant volume that includes the vessel and/or on an organ tissue.

In a localized-DCE scan, less information is required and derived relative to a volumetric-DCE scan. As such, a localized-DCE scan can have lower spatial resolution, lower temporal resolution (e.g., fewer time frames), narrower axial coverage, and/or higher image noise, relative to a volumetric-DCE scan. Furthermore, a localized-DCE scan may, overall, have an image quality which renders the images non-diagnostic. Thus, the localized-DCE scan protocol can be performed with lower x-ray radiation dose and contrast agent dose relative to a diagnostic volumetric-DCE protocol.

The following describes an approach for generating fluid volume maps based on contrast enhanced spectral volume image data and a set of localized-DCE images such as a set of contrast enhanced axial series scan image, etc. As described herein, a fluid volume map can be determined for blood, lymph, and/or other fluids in a subject. For example, with respect to blood volume, this includes arterial and/or capillary blood volume maps. Generally, the volume scan is a diagnostic scan whereas the time-series scan can be a lower dose, non-diagnostic scan. This approach reduces patient dose and provides larger axial coverage, relative to a CT perfusion scan, and does not require image registration between multiple time frames.

FIG. 1 schematically illustrates an example imaging system 100 such as a computed tomography (CT) system, which is configured for spectral and non-spectral (or conventional) imaging. The imaging system 100 includes a generally stationary gantry 102 and a rotating gantry 104, which is rotatably supported by the stationary gantry 102. The rotating gantry 104 rotates around an examination region 106 about a longitudinal or z-axis. A radiation source 108, such as an x-ray tube, is supported by the rotating gantry 104 and emits poly-energetic/chromatic radiation. A collimator (not visible) collimates the radiation beam to produce a generally cone, fan, wedge, cone or otherwise shaped radiation beam that traverses the examination region 106.

A one or two dimensional radiation sensitive detector array 110 detects radiation that traverse the examination region 106. The illustrated radiation sensitive detector array 110 includes spectral detectors. In one instance, the spectral detectors include one or more scintillator layers optically coupled to two or more photosensors. In a dual photosensor configuration, lower energy photons generally are absorbed in scintillators layers or the portion of a single layer closer to the incident radiation, and higher energy photons generally are absorbed in scintillators layers or the portion of a single layer farther away from the incident radiation. A photosensor receive light from the respective scintillator layer or portion of the layer and produces an output signal indicative of the energy of the incident radiation.

An example of a multi-layer spectral detector is the dual-layer or double decker spectral detector described in U.S. Pat. No. 7,968,853 B2, filed Apr. 10, 2006, and entitled “Double Decker Detector for Spectral CT,” which is incorporated herein by reference in its entirety. In a variation, at least one of the spectral detectors 110 includes a solid-state spectral detector. For instance, at least one of the spectral detectors 110 can include a direct conversion material such as cadmium telluride (CdTe), cadmium zinc telluride (CdZnTe, or CZT), etc. The output signal of the direct conversion material for detected radiation is indicative of the energy of the detected radiation. Other spectral or energy resolving detectors are also contemplated herein.

Additionally or alternatively, the spectral imaging system 100 may include two or more x-ray tubes configured to emit radiation having different mean spectrums and/or a single x-ray tube configured to controllably switch between at least two different emission voltages during scanning.

A reconstructor 112 reconstructs the signal from the radiation sensitive detector array 110 via spectral and non-spectral reconstruction algorithms. A suitable spectral reconstruction algorithm decomposes the energy-resolved signals into various energy dependent components. For example, in one instance a detected energy-resolved signal is decomposed into a Compton component image, a photo-electric component image, and one or more k-edge component images representative of one or more k-edge materials, e.g., such as iodine, in a contrast material. With embodiments in which the contrast material includes a k-edge material, the k-edge component generally reflects the contrast material, whereas the Compton and photo-electric components are used to reflect the anatomical structure.

It is to be appreciated that a maximum likelihood and/or other decomposition technique may be used. An example decomposition approach is described in application serial number PCT/IB2007/055105, filed on Dec. 14, 2007, which claims the benefit of provisional application serial number EP 06126653.2, filed on Dec. 20, 2006, both of which are incorporated in their entirety herein by reference. A suitable non-spectral reconstruction algorithm includes filtered-back projection (FBP), etc. Alternatively, the non-spectral reconstruction algorithm may combine the spectral component images, thereby forming a single non-spectral image.

In another variation, the acquired data of each energy range among a plurality of x-ray energy ranges is reconstructed independently and a post-processing spectral analysis is performed. An example of this approach is discussed in U.S. Pat. No. 7,778,380, filed Aug. 16, 2006, and entitled “Data handling and analysis in computed tomography with multiple energy windows,” which is incorporated in its entirety herein by reference.

A subject support 114 such as a couch supports a subject or an object in the examination region 106.

An injector 115 is configured to inject a contrast material(s) contrast enhanced imaging procedures. The illustrated injector 115 is controlled by a console 116, which may trigger or invoke in the injector 115 to administer the contrast material in coordination with scanning such that contrast uptake and enhancement by tissue of interest is scanned. A contrast agent can additionally or alternatively be manually administered by a clinician or the like. Where the contrast agent is manually administered, the injector 115 can be omitted. With further respect to the contrast agent, generally, a suitable contrast material may include an iodine and/or other material with a k-edge in the diagnostic x-ray energy (e.g., 20-140 keV) and distinguishable from the photoelectric and Compton components.

A computer serves as the console 116 and includes a human readable output device such as a monitor or display and an input device such as a keyboard and mouse. Software resident on the console 116 allows the operator to interact with the scanner 100 via a graphical user interface (GUI) or otherwise. This includes selecting a scan protocol(s) that generates image data, which can be further processed to determine fluid volume maps. This includes a scan protocol(s) for a contrast enhanced spectral volume scan and, optionally, a localized-DCE series scan. The localized-DCE scan can be a lower dose scan relative to a volumetric DCE scan and even relative to the volume scan and may have an image quality which renders the images non-diagnostic such as with very low spatial resolution or with a very narrow axial coverage or with a few scanned time frames. Where the localized-DCE series scan is performed with a different imaging system, selection of the localized-DCE scan protocol can be omitted.

Example parameters for a localized-DCE series scan, for an average adult patient, include a series of 10 to 20 successive axial scans, with a delay of 2 to 3 seconds between scans, and each with acquisition and reconstruction slice thickness in a range from 5 to 10 millimeters (mm), with relatively low resolution, tube current in a range of 20 to 50 milliAmpheres-seconds/pitch (mAs/pitch) per scan, administered iodine contrast agent total amount in a range of 5 to 15 cubic centimeters (cc), with an injection rate of approximately 5 cc/second, and an injection duration in a range of 1 to 3 seconds. The above is a non-limiting example, and it is to be understood that other parameters are contemplated herein. An example of such a scan is a time lapse CT (TLCT) scan, a conventional series scan, etc.

Example parameters for a suitable contrast enhanced spectral volume scan, for an average adult patient, include a single helical scan with full organ (e.g., the heart, lungs, etc.), regional (e.g., chest, abdomen, etc.) or whole body coverage, reconstruction slice thickness in a range from 0.6 to 3.0 mm, tube current in a range of 50 to 300 milliAmpheres-seconds (mAs), administered iodine contrast agent total amount in a range of 30 to 50 cc, with an injection rate of approximately 5 cc/sec, and an injection duration in a range of 6 to 10 seconds. The above is a non-limiting example, and it is to be understood that other parameters are contemplated herein.

The localized-DCE series scan can be used to plan the contrast enhanced spectral volume scan, e.g., used to select an optimal time for the volume scan (relative to contrast agent injection time), etc. Where the localized-DCE series scan is not used to plan the contrast enhanced spectral volume scan, the contrast enhanced spectral volume scan can be performed first or second. The localized-DCE series scan and the contrast enhanced spectral volume scan can both be performed with the imaging system 102, or the localized-DCE series scan can be performed with another imaging system. The resulting data of the two scans need not be registered.

A fluid volume map determiner 118 determines fluid volume maps. As described in greater detail below, the fluid volume map determiner 118 determines volumetric quantitative fluid (e.g., blood, lymph, etc.) volume maps based on the contrast enhanced spectral volume image data and the set of localized-DCE images of one or more organs of interest (e.g. liver, spleen, kidney, pancreas, lymph node, etc.) and at least one tubular structure (e.g., a blood vessel, a lymphatic vessel, etc.) routing or carrying the fluid to the one or more organs of interest. In the illustrated example, these two scans can be performed with the imaging system 102. The fluid volume map determiner 118 can also determine other maps such as a flow per volume map, etc.

It is to be appreciated the fluid volume map determiner 118 can be implemented by one or more computer processors (e.g., a central processing unit or CPU, a microprocessor, a controller, etc.) executing one or more computer readable instructions stored, encoded, embedded, etc. on computer readable storage medium (which excludes transitory medium) such as physical computer memory. Additionally or alternatively, at least one of the computer readable instructions executed by the one or more computer processors is carried by transitory medium such as a signal or a carrier wave. Furthermore, the fluid volume map determiner 118 can be part of the console 116 and/or separate therefrom and part of separate computing system and/or distributed across two or more computing systems.

FIG. 2 schematically illustrates an example of the fluid volume map determiner 118. For sake of brevity and explanatory purposes, the following is described with respect to determining volumetric quantitative blood volume maps. However, invention is not so limited, and can generate maps for other fluids, such a lymph, etc. Generally, the lymphatic system parallels the circulatory system. However, with the lymphatic system, the tubular structure would be the lymphatic vessels, the organs would be the lymphatic organs, and the fluid would be lymph.

A region of interest (ROI) identifier 202 identifies one or more ROIs in the series of localized-DCE images. In the illustrated embodiment, each ROI is for a single or an average of several voxels and/or sub-portions of voxels. Automated and/or manual (with user interaction) approaches can be used to identify the ROIs. In one non-limiting example, the ROIs include at least a sub-portion of an input or reference artery (e.g. the aorta), which routes blood to one or more organs of interest, and at least a sub-portion of the one or more organs of interest (e.g. liver, spleen, kidney, etc.).

A time attenuation curve (TAC) determiner 204 determines a respective time attenuation curves (TACs) for each of the identified ROIs. A curve fitter 206 creates model curves for the artery and the one or more organs of interest based on a predetermined model and the TACs for the ROI's. In one instance, this includes curve-fitting the measured ROI TACs to find an average model curves for the artery and the one or more organs of interest. In one example, the fitted function relates to a pre-determined model of blood flow, such as Gamma-variate curves with parameters, which are constrained by the model.

Briefly turning to FIG. 3, example fitted curves 302 and 304 created by fitting gamma-variate curves to the measured TACs for ROI's respectively for the aorta and the spleen are illustrated. Curve 302 ignores the later recirculation in the aorta. Examples of the fitted functions are shown in EQUATIONS 1 and 2:

$$EQUATIONS 1 \text{:}$$

$$y ( t ) = {{y}_{max 1} ( \frac{t - {t}_{0}}{{t}_{max 1} - {t}_{0}} )}^{α} \cdot exp ( α ( 1 - \frac{t - {t}_{0}}{{t}_{max 1} - {t}_{0}} ) ) , t > 0 ,$$

for the aorta, and

$$EQUATIONS 2 \text{:}$$

$$y ( t ) = {{y}_{max 2} ( \frac{t - {t}_{0}}{{t}_{max 2} - {t}_{0}} )}^{α} \cdot exp ( α ( 1 - \frac{t - {t}_{0}}{{t}_{max 2} - {t}_{0}} ) ) , t > 0 ,$$

for the spleen. Since both of the TACs are related to the same blood circulation and contrast agent bolus, the parameters t₀ and α can be set to the same or different value for both. In FIG. 3, the fit results are: t0=3.6, alpha=1.62, ymax1=115.5, ymax2=18.6, tmax1=5.30, tmax2=8.77.

Returning to FIG. 2, a flow estimator 208 estimates a general blood flow model for the artery and the one or more organs of interest for (e.g., a hypothetical situation of) 100% blood volume. In general, this is done only for the artery and other blood vessels. In the one or more organs of interest, the blood volume varies within different locations and it is typically much less than 100%. As such, true values should be found for the blood volume in the one or more organs of interest.

In one instance, the flow estimator 208 estimates the model by estimating a convolution time window based on the localized-DCE series scan injection time window and the contrast enhanced spectral volume scan injection time window. For example, the convolution time window can be a rectangular function with a width that is equal to a difference between the injection time duration of the localized-DCE series scan injection time window and the injection time durations of the contrast enhanced spectral volume scan injection time window.

The flow estimator 208 then convolves the convolution time window with the TACs, which estimates TACs corresponding to the injection of the contrast enhanced spectral volume scan. Next, the blood flow estimator 208 multiplies the estimated organ TAC by a factor that equalizes the area under the curve of the estimated organ TAC to that of the estimated artery TAC. This will simulate a hypothetical situation that the organ has 100% blood volume.

An iodine map generator 210 generates quantitative volumetric iodine maps based on the contrast enhanced spectral volume image data. This includes a map for the artery and the one or more organs of interest. A generated iodine map is specific only to the time of the scan and therefore, alone, does not indicate blood volume. There is no need to calibrate the iodine map to the iodine concentration since the normalization by the input artery data will give pure units.

A volume determiner 212 determines a volumetric fluid volume map based on the iodine map and the estimate of the general blood flow model. In one instance, this includes calculating a first ratio of the 100% blood volume organ curve to the artery curve and calculating a second ratio of the organ iodine map value to the artery map value. The first ratio is calculated for each time point, and the second ratio is calculated for the specific measurement time point and each voxel (or other location selection in the volume).

The volume determiner 212 further calculates a third ratio, which is a ratio of the second ratio to the first ratio. This includes, for each voxel, dividing the second ratio by the first ratio. The time correspondence between the measurement and the model curves are done by the recorded times relative to the injection starting point. Note that if the contrast enhanced spectral volume scan is done on a region where no main artery is presented, the quantitative properties of the iodine map can be used along with the amount of injected contras agent to derive blood volume results.

A rendering engine 214 visually presents the blood volume maps via a display, such as a display of the console 116 (FIG. 1) and/or other display. This can be achieved through techniques such as color fusion with the contrast enhanced spectral volume image data and/or alone as an independent image volume.

Variations are discussed next.

In a variation, a different time point for each slice in the volume scan can be calculated, taking into account the translation of the subject support 114 as a function of time. This may lead to a higher accuracy.

In another variation, flow models for two or more different ROIs at different locations in the organ of interest can be used. Likewise, this may lead to a higher accuracy.

In another variation, the blood volume calculation may be based on each voxel or group of voxels using a smoothed TLCT image

In another variation, image data from a non-spectral CT scan is used. In this variation, two non-spectral CT scans are required and, the image data from the two non-spectral CT scan would have to be registered.

In another variation, image data generated by other imaging modalities, such as MR, PET, etc., in which a dynamic of contrast agent or tracer can be measured and can be used to determine the blood volume maps.

In another variation, the blood volume is determined based on a two-circulation flow pattern. For example, certain organs have dual-circulation system. Non-limiting examples include the liver with arterial and hepatic portal phases and the lungs with pulmonary and arterial phases. With such organs, it is possible to derive the relative blood volume corresponding to each of the two circulation flow patterns by using two successive contrast enhanced spectral volume scans.

For example, a first local iodine map value for a first measurement, for a particular location, from first contrast enhanced spectral volume image data from a first contrast enhanced spectral volume scan can be expressed as shown in EQUATION 3:

m₁=f₁·a(t₁)+f₂·b(t₁), and EQUATIONS 3:

a second local iodine map value for a second measurement, for the same particular location, from second contrast enhanced spectral volume image data from a second different contrast enhanced spectral volume scan can be expressed as shown in EQUATION 4:

m₂=f₁·a(t₂)+f₂·b(t₂), EQUATIONS 4:

where m₁ is the first local iodine map value, m₂ is the second local iodine map, a(t) is the modeled arterial flow (for 100% blood volume), and b(t) is the modeled hepatic portal flow (for 100% blood volume) where the liver is the organ of interest.

The two required relative blood volume maps can then be derived by solving the two linear EQUATIONS 3 and 4. a(t) and b(t) are already normalized by the measured aorta ROI. The image data of the two scans may be spatially registered, which may improve the accuracy of the blood volume maps.

FIGS. 4 and 5 respectively show spectral image data based iodine maps of a dual-circulation organ (liver) and a single-circulation organ (spleen). The two maps are taken from two different scans in different times.

FIG. 6 illustrates a method for determining a volume map based on contrast enhanced spectral volume image data and a set of localized-DCE images.

It is to be appreciated that the ordering of the acts of these methods is not limiting. As such, other orderings are contemplated herein. In addition, one or more acts may be omitted and/or one or more additional acts may be included.

At 602, a localized-DCE series scan of a subject is performed, producing a set of localized-DCE images. The contrast injection start time and/or duration is recorded. Where the scan was already performed, this act instead includes obtaining the set of localized-DCE images. As discussed herein, this scan can be a TLCT scan or other scan with a lower dose that results in non-diagnostic quality images.

Generally, this scan is used to measure time attenuation curves for only ROIs at a selected axial position of the subject support 114. As such, a relatively low radiation dose and small amount of contrast agent are sufficient. An example of such a scan is a time lapse CT (TLCT) scan. Furthermore, the resulting set of localized-DCE lower dose images can be used to plan the contrast enhanced spectral and/or other volume scan.

At 604, a contrast enhanced spectral volume scan of the subject is performed, producing contrast enhanced spectral volume image data. The contrast injection start time and/or duration is recorded. Where the contrast enhanced spectral volume scan was already performed, this act instead includes obtaining the contrast enhanced spectral volume image data. The localized-DCE images can be used to select the optimal time for performing the contrast enhanced spectral volume scan relative to the contrast agent injection time.

In a variation, act 604 is performed before act 602.

At 606, ROIs that include at least a sub-portion of an organ of interest and at least one artery, which routes blood to the at least one organ of interest, are identified.

At 608, TACs for the ROIs of the at least one organ of interest and for the ROI of the artery are determined.

At 610, model TAC curves are created for the artery and the at least one organ of interest. In one instance, this includes curve-fitting the measured ROI TACs to predetermined functions to find an average model curves for the artery and the at least one organ of interest, as describe herein or otherwise.

At 612, a model for the general blood flow of the artery and the at least one organ of interest, for the hypothetical situation of having 100% blood volume, is calculated, as described herein and/or otherwise.

At 614, iodine maps are generated based on the contrast enhanced spectral volume image data, as discussed herein or otherwise.

At 616, for each time point of the series scan, a first ratio between the model value of the at least one organ of interest at 100% blood volume and the model value of the artery at 100% blood volume is calculated, as described herein and/or otherwise.

At 618, for the specific measurement time point of the volume scan and for each voxel (or other location selection in the volume), a second ratio between the at least one organ of interest iodine map value and the artery map value is calculated, as described herein and/or otherwise.

At 620, a true local value of a volumetric blood-volume map is generated based on a ratio of the second ratio (act 618) to the first ratio (act 616), as described herein and/or otherwise.

At 622, the volumetric blood volume map is visually presented. This may include visually presenting the maps via color fusion with the contrast enhanced spectral volume image data, alone as an independent image volume, etc.

The above may be implemented by way of computer readable instructions, encoded or embedded on computer readable storage medium (which excludes transitory medium), which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method, comprising:
obtaining image data that includes contrast enhanced spectral image data from a contrast enhanced spectral volume scan of a subject;
obtaining images that include a set of localized dynamic contrast enhanced images from a localized dynamic contrast enhanced series scan of the subject; and
determining a fluid volume map based on the image data and the images.

2. The method of claim 1, wherein the fluid includes one of lymph or blood.

3. The method of claim 1, further, comprising:
determining a first time attenuation curve for a first region of interest identified for at least one organ of interest and a second time attenuation curve for a second region of interest of a tubular structure routing the fluid to the at least one organ of interest; and
creating a first model time attenuation curve for the at least one organ of interest and a second model time attenuation curve for the tubular structure by fitting the first and second time attenuation curves to find average model curves for the tubular structure and the at least one organ of interest; and
determining the fluid volume map based on the first and second model time attenuation curves.

4. The method of claim 3, wherein the tubular structure is one of a blood vessel or a lymph node.

5. The method of claim 3, further comprising:
scaling the first and second model time attenuation curves to 100% fluid volume; and
determining the fluid volume map based on the scaled first and second model time attenuation curves.

6. The method of claim 3, further comprising:
calculating, for each time point of the series scan, a first ratio of the first model time attenuation curve to the second model time attenuation curve; and
determining the fluid volume map based on the first ratio.

7. The method of claim 6, further comprising:
obtaining a first iodine map of the at least one organ of interest and the tubular structure;
calculating, for the measurement time point of the volume scan and for each voxel, a second ratio of an iodine map value of the at least one organ of interest to the map value of the tubular structure; and
determining the fluid volume map based on the second ratio.

8. The method of claim 7, further comprising:
generating the first iodine map of the at least one organ of interest and the tubular structure based on the contrast enhanced spectral volume image data.

9. The method of claim 7, further comprising:
calculating a third ratio of the second ratio to the first ratio; and
determining the fluid volume map based on third second ratio.

10. The method of claim 9, wherein the third ratio is indicative of a local value of volumetric fluid-volume map.

11. The method of claim 7, further, comprising:
obtaining a second iodine map of the at least one organ of interest and the tubular structure; and
determining the fluid volume map based on the first and second iodine maps and the modeled fluid flow.

12. The method of claim 11, further comprising:
generating the second iodine map of the at least one organ of interest and the tubular structure based on the second different contrast enhanced spectral volume image data from a second different contrast enhanced spectral volume scan.

13. The method of claim 12, wherein the at least one organ of interest includes a structure with dual circulation fluid system.

14. The method of claim 1, further, comprising:
visually presenting the fluid volume map.

15. A computing system, comprising:
a computer readable storage medium with computer readable instructions for a fluid volume map determiner that determines a volumetric fluid volume map based on contrast enhanced spectral volume image data from a contrast enhanced spectral volume scan and a set of localized dynamic contrast enhanced images from a localized dynamic contrast enhanced series scan; and
a processor that implements the instructions and determines the volumetric fluid volume map.

16. The computing system of claim 15, wherein the fluid volume map determiner includes:
region of interest identifier that identifies, with respect to the set of localized dynamic contrast enhanced images, a first region of interest of a structure of interest and a second first region of interest of a tubular structure that routes a fluid to the structure of interest;
a time attenuation curve determiner that determines a first time attenuation curve for the first region of interest and a second time attenuation curve for the second region of interest;
a curve fitter that fits the first and second time attenuation curves to respective predetermined functions to determine first and second average model curves respectively for the first region of interest and the second region of interest;
a flow estimator that estimates a first model time attenuation curve for the first region of interest and a second model time attenuation curve for the second region of interest for 100% fluid volume based on the first and second fitted curves;
an iodine map generator that generates an iodine map, for the at least one organ of interest and the tubular structure, based on the contrast enhanced spectral volume image data;
a volume map determiner that determines a volume map based on the first and second model time attenuation curves and the iodine map; and
a rendering engine that visually displays the volume map.

17. The computing system of claim 15, wherein the volume map determiner:
calculates, for each time point of the series scan, a first ratio of the first model time attenuation curve to the second model time attenuation curve;
calculates, for the measurement time point of the volume scan and for each voxel, a second ratio of an iodine map value of the at least one organ of interest to the map value of the tubular structure; and
determines the volume map based on the first and second ratios.

18. The computing system of claim 17, wherein the volume map determiner:
calculates a third ratio of the second ratio to the first ratio, wherein the third ratio is indicative of a local value of volumetric fluid-volume map.

19. The computing system of claim 15, wherein the fluid includes one of lymph or blood and the tubular structure is one of a blood vessel or a lymph node.

20. A computer readable storage medium encoded with computer readable instructions, which, when executed by a processor of a computing system, causes the processor to:
determine a volumetric blood volume map based on contrast enhanced spectral volume image data generated in response to contrast enhanced spectral volume scan of a subject and images and a set of localized dynamic contrast enhanced images generated in response to a localized dynamic contrast enhanced series scan of the subject.
